(19) United States
(12) Patent Application Publication
(10) Pub. No.: US 2012/0302863 A1
(43) Pub. Date: Nov. 29, 2012

(54) Medical Procedure Localizing Aid

(75) Inventor: Colin O'Neill, West Vancouver (CA)

(73) Assignee: AEOS BIOMEDIAL INC., Vancouver, BC (CA)

(21) Appl. No.: 13/522,624

(22) PCT Filed: Nov. 22, 2010
(86) PCT No.: PCT/CA2010/001881

§ 371 Date: Jul. 17, 2012

Related U.S. Application Data
(60) Provisional application No. 61/337,265, filed on Feb. 2, 2010.

(51) Int. Cl. A61B 6/00

(52) U.S. Cl. 600/407

(57) ABSTRACT

A medical procedure localization aid produces reference marks on both the patient and the medical imaging scan. The aid is defined by a substrate sheet having indicia on one side that is opaque to medical imaging radiation and indicia on a second side that is transferable to a patient. The indicia on the first side is displayed on the scan image and the indicia on the second side is imprinted on the patient. By visualizing the location of a target on the scan image relative to the indicia on the scan image and comparing that with indicia on the patient, a medical professional may reliably locate where a medical procedure should be performed.

### FIELD OF THE INVENTION

This invention relates to apparatus and methods used in the medical field, and more specifically to apparatus and methods for medical procedure localization.

### BACKGROUND

A medical imaging scan, such as an X-ray, computerized tomography, position emission tomography, and/or nuclear magnetic resonance imagining, is commonly utilized first to determine if a surgical procedure is necessary. The medical professional such as a doctor, surgeon, radiologist, nurse, technician, veterinarian or clinical researcher relies on this image to view the precise internal information of the patient. If the medical image shows irregularities, the medical professional may then conclude that a medical procedure is necessary. When the precise area of interest is determined on the medical image, the medical professional will have to perform their procedure directly above this area. Examples of these various procedures include surgical incisions to treat fractures, spinal and thoracic lesions, the removal of foreign bodies and biopsies. These medical procedures are not limited to human subjects, but can also include other animals or cadavers for veterinary or clinical research procedures.

The medical professional relies on an initial medical image for guidance to the target location. Intraoperative scanning, such as fluoroscopy, is commonly utilized during a medical procedure when sequential images are needed. These medical images will show the exact internal point of interest. However, the medical image will show no visual of the patient's external anatomy, which would be useful to localize exactly where the procedure should be performed—the word patient meaning herein any subject a medical professional can perform a procedure on, examples including humans, animals, cadavers or any other form of a test subject. The medical professional will rely on palpation of anatomical landmarks to try and perform their procedure directly above their targeted area of interest. This method is prone to inaccuracy and often requires an extension of the incision or working awkwardly through an angled trajectory. In some instances, the medical professional may even accidentally perform the procedure on the wrong area because the site of the incision was inaccurate.

Inventors have created several types of aids to assist with medical imaging guidance. U.S. Pat. No. 6,333,970 to LeMaitre et al (2001) discloses an adhesive with radiopaque indicia in the form of a linear graduated pattern. The adhesive is placed on the patient before the scan and items underneath the skin can be sized and their location determined. However, when the patient enters the operating room, the adhesive is removed along with the reference marks on the body. The locational marks on the scan are not useful if they can no longer be referenced to on the body. Therefore, LeMatire's tape is primarily used to aid diagnostics of an x-ray image.

U.S. Pat. No. 4,506,676 to Duska (1985) utilizes a radiopaque dotted line on an adhesive tape that will guide the medical professional to the area of interest on the x-ray image. This device will show as a line on the x-ray image, but does not provide direct locational guidance on the body when the patient enters the operating room.

U.S. Pat. No. 5,848,125 to Arnett (1998) also attempts to bring locational information to an x-ray image by placing a small metallic pellet underneath an adhesive. The pellet gives doctors a reference point to look for on the x-ray image, where they then note and estimate the landmark closest to the pellet. When it comes time to perform the procedure, they will again estimate the distance from that landmark to the area of interest. The pellet also obstructs the medical professional from marking the skin of the incision area with their surgical pen. The pellets must be removed, and thus rendered useless, if the medical professional marks their area of interest.

U.S. Pat. No. 5,193,106 to DeSena (1993) discloses radiopaque stickers with flat shapes formed thereon including a circle, the outline of a square, and the outline of a triangle. This device is limited to small shapes for the foot and must be removed during the procedure.

Inventors have also proposed devices to aid in making more accurate incisions. U.S. Pat. No. 6,972,022 to Griffin (2005) discloses a skin-marking device that marks skin with a radiopaque substance, a fluorescent composition, a non-magnetic hydrogel for nuclear magnetic resonance imaging, a sterilizable gel ink, a combination of any of these, and a mixture of any of these. Using a free hand pen to mark the skin for locational purposes is messy, limits the precision of designs and the accuracy of consistent spacing. This method is time consuming and does not provide the ability to create standard guides for the technicians performing the medical scans. The marks on the skin would also be opaque to follow up medical imaging scans. The medical professional will have to remove the markings if a clean scan is needed, thus rendering the pen markings useless.

U.S. Pat. No. 5,323,452 to Russell et al. (1999) discloses an alternate marker system for radiography which includes an elongate base tape, a bendable, fabric covered wire containing a material that is radiopaque, and a continuous row of adhesive pads fixedly aligned along the wire. The adhesive pads and the carried radiopaque wire are manually removable from the base tape together with the wire for releasable adherence to a subject. When imaged, the wire will show up as a continuous line. However, if the line is far away from the target of interest, localizing it on the skin will be prone to inaccuracies. If the line is over top an area of interest, the medical professional will still have to visually estimate where along the line the target is since there are no reference marks along the line. The wire would also have to be removed before the patient enters the operating room or before the surgeon begins performing the procedure.

U.S. Pat. No. 7,677,801 to Pakzaban (2010) discloses a device that utilizes a crosshair projected on a patient's back by lasers. Attached to the device are radiopaque cables to assist with targeting the correct vertebrae. This device is expensive and must be sterilized before every use. It is also time consuming. If the device is placed over top of the incorrect vertebrae, it must then be moved up or down the patient's back and a subsequent scan must be taken. This process must be repeated until the medical professional has the laser cross hair directly over the targeted vertebrae.

Other inventors have proposed aids to assist with locational guidance for inserting biopsy needles into a patient's body. U.S. Pat. No. 4,860,331 to Williams et al. (1989) discloses an adhesive tape structure with a plurality of radiopaque vertical lines, with biopsy needle holes formed between the parallel vertical lines. This structure is said to be useful during computerized tomography scans to aid in locating the appropriate position to insert a biopsy needle. This device cannot be removed because there will be no reference for the medical professional to know where to insert their needle. The accuracy of this device is limited because the medical professional is restricted to insert the needle only through the holes in the device. The hole may or may not be directly above the optimal entry point for biopsy needle insertion.

U.S. Pat. No. 6,714,628 to Broyles et al. (2004) expands upon the '331 patent to Wiliam et al., described above, with an adhesive with a plurality of radiopaque vertical lines, with vertical cutouts between the radiopaque lines. This device gives a larger area to insert the biopsy needle into, but still leaves a chance that the cut out area is not directly above the optimal entry point for biopsy needle insertion. This device has limited practical surgical use, and an inconvenient method of imprinting any reference marks on the body.

Lastly, inventors have proposed a device that will mark the skin with ink. U.S. Pat. No. 5,743,899 to Zinreich et al. (1997) discloses an adhesive material with an ink pattern deposited on an adhesive. When the tape is applied to the skin, the ink markings will transfer to the skin. This device will leave a reference on the body, but is missing the reference marks projected on the medical imaging scan. The medical professional will not have reference marks on both the scan image and the body.

The patents described briefly above demonstrate that there is a distinct need for apparatus and methods that allow non-invasive and accurate medical procedure localization that allows a medical professional to reliably locate on a patient's body the precise target location for performing a medical procedure.

### SUMMARY OF THE INVENTION

The apparatus and method of the present and illustrated inventions are based on a device that defines an improved means and method of non-invasively locating a procedure site on a patient prior to surgery. The inventive apparatus may be used in connection with numerous types of medical imaging scans, such as, but not limited to: X-rays, computerized tomography, position emission tomography, ultrasound and nuclear magnetic resonance imagining. The aid will produce reference marks on both the patient and the medical imaging scan. The aid is utilized in two phases. The first phase involves the aid attached to the body before a medical imaging scan. The resulting image from the scan shows a visual of both the internal anatomy and the opaque indicia from the aid itself—the word opaque meaning herein a material that would appear on the medical image. The second phase involves the removal of the aid. Upon removal of the aid an identical or correlating visual mark of the indicia will be on the body and may or may not be of a marking material opaque to the medical imaging scan. As a result, the medical professional may reliably use the combination of the medical imaging scan with the indicia visible on the patient's body to accurately locate the target site for the indicated medical procedure. Thus, the medical imaging scan allows the medical professional to identify the location of the target for a procedure, with that target being locatable in relation to the opaque indicia that is visible on the scan. The same indicia are imprinted on the patient's body. The medical professional may compare the two to accurately determine where the procedure should be carried out.

Accordingly, several objects and advantages of the invention are: to provide visual indicia marks on both the medical image and on the patient's body; to provide a quicker and more accurate means of surgical localization; to reduce the need of multiple fluoroscopy scans and thereby reduce radiation exposure to both patients and medical staff; to provide optimized location designs which are pre-drawn; to provide markings days before the procedure or to be utilized intraoperatively; and to provide a visual marking on the patient's skin to allow for more accurate/precise suturing by re-aligning these marks back to its original design.

### BRIEF DESCRIPTION OF THE DRAWINGS

For a better understanding of the present invention, in conjunction with other objects, features, and advantages, references should be made to the following description of a preferred embodiment. The preferred embodiment should be read with reference to the appended drawings, in which:

FIG. 1 is a schematic top view of a first illustrated embodiment of the medical procedure localizing aid according to the present invention positioned on a patient's back with the patient in a prone position.

FIG. 2 is a top perspective view of the embodiment of the medical procedure localizing aid shown in FIG. 1, illustrating the protective backing liner being peeled away from the main body of the aid.

FIG. 3 is a top perspective view of the embodiment of the medical procedure localizing aid shown in FIG. 1, illustrating the transferable marking mechanism behind on the patient as the main body of the aid is peeled away from the patient's body.

FIG. 4 is a schematic view of an exemplary x-ray image of a patient's body (in this image the patients hips and spine) showing the gridlines imprinted on the x-ray from the opaque material deposited on the aid.

FIG. 5 is a schematic top view of the upper torso region of a patient shown in a prone position, illustrating the markings left behind on the patient's body after the medical procedure localization aid according to the present invention has been removed.

FIG. 6 is an x-ray image of the upper torso region of the patient shown in FIG. 5, illustrating the opaque material from the medical procedure localization aid as it is seen on the x-ray film.

FIG. 7 is a top plan view of one embodiment of a medical procedure localization aid according to the present invention, illustrating one possible indicia pattern, in this case a grid pattern.

FIG. 8 is a cross sectional view of a portion of an alternative embodiment of a medical procedure localization aid according to the present invention.

FIG. 9 is a cross sectional view of a portion of yet another alternative embodiment of a medical procedure localization aid according to the present invention.

FIG. 10 is a top plan view of the outer layer of the embodiment of the medical procedure localization aid according to the present invention and shown in FIG. 9.

FIG. 11 is a top plan view of one layer of the embodiment shown in FIG. 9.

FIG. 12 is a top plan view the outer layer shown in FIG. 10 superimposed over the layer shown in FIG. 11.

### DESCRIPTION OF PREFERRED EMBODIMENTS

In a most preferred embodiment, the medical procedure localizing aid 10 according to the present invention is a sterile, flexible adhesive-backed sheet or substrate having opposed top and bottom surfaces. As a naming convention, for purposes herein the “top” or “upper” surface of the localization aid is the surface of the sheet that is exposed and faces away from the patient when the localization aid is in place. The “bottom” or “lower” surface is then the opposite side of the sheet—that is, the side of the sheet on which an adhesive material has typically been applied and which is applied to the patient's skin when the localization aid is in place. The actual geometric configuration of the sheet can range from a variety of different shapes and sizes. This can range from a large area to cover the chest and back to small narrow strips for fingers and toes, or any other convenient size or shape. The top surface will suspend indicia that is opaque to medical imaging scans—as noted above, as used herein the word “opaque” means a material that will visually appear on a medical imaging scan. Numerous types of opaque materials are contemplated for use with the present invention, the actual compounds comprising the opaque material often depending upon the type of medical imaging scan for which the localization aid is designed. Thus, the opaque indicia may be of a type that is radiopaque for localization aids that are intended for use with x-radiation. Examples of compounds suitable for opaque materials for use with x-radiation include, but are not limited to, barium sulphate, lead, tantalum, triphenylbismuth or copper. The opaque indicia may also be of a non-magnetic hydrogel, such as vitamin K, for the use in nuclear magnetic resonance imaging. Other compounds are also contemplated and the invention is not limited to any particular type of material used for the opaque material.

The opaque indicia will be placed on the top surface of the sheet in the form of a locational reference pattern. The locational reference pattern is a design in a logical format used to quickly and efficiently locate a specific space in a larger area. These patterns can range from, but are not limited to: a grid, dots, cross hatches, circles, graduated linear pattern, a combination of any of these, or any other logical design that will assist with locating a precise area within a space. The design of the reference patterns may also be in a graduated linear pattern, such as a ruler with gradations in either alpha or numeric characters, which allow a medical professional to, for example, determine the size of objects shown in the medical image. The resolution of these patterns and indicia used with the patterns may range from very high to very low; a high resolution pattern will have lines or gradations that are close together. These types of patterns can be used for small, superficial targets. On the other hand, a low resolution pattern will have lines or gradations that are further apart. These types of patterns are useful for relatively larger, deeper below-the-surface targets.

A marking mechanism is located on the bottom surface of the sheet. Included in the marking mechanism is a transferable marking material, such as an ink or other compound that is designed to transfer from the sheet to the patient so that an aligned identical or logically correlatable pattern as the opaque reference pattern on top surface indicia is imprinted onto the patient's skin. The related pattern imprinted onto the patient's skin has identical locational information, but may be optimized for skin imprinting by having thicker lines and/or larger symbols. The marking mechanism may imprint the pattern on the patient in a variety of different methods. Examples can include, but not be limited to, an ink stamp, a temporary tattoo or an etched pattern filled with ink.

An adhesive material is deposited on the bottom surface of the sheet. The adhesive does not interfere with transfer of the marking mechanism to the patient, as described below. A removable protective backing sheet will be in contact with the adhesive to protect, store, and prevent the aid from being adhered inadvertently to itself or other objects. The removable protective backer can be coated with a wax or silicon material to preserve the adhesiveness while allowing for easy separation from the aid. Moreover, the adhesive used is preferably an adhesive that will not cause adverse topical reactions in most patients.

In a preferred method of use, the medical professional or medical assistant first removes the protective backing material from the localization aid and then applies the aid to the patient's body in the appropriate area of interest. As used herein, the term “medical professional” includes a wide variety of workers in the medical and veterinary fields, for example, doctors, surgeons, radiologists, nurses, technicians, veterinarians and clinical researchers. The adhesive material on the “bottom” side of the sheet defining the localization aid causes the aid to adhere to the patient's body in the desired location. The patient will then proceed to have their medical imaging scan done. As noted previously, the medical imaging scans with which the present invention may be used include, but are not limited to, X-ray, computerized tomography, position emission tomography, and nuclear magnetic resonance imagining. Further, the patient may even be in the operating room where the medical professional can utilize the device throughout the procedure intraoperatively using fluoroscopy. Accordingly, the localization aid of the present invention is not limited to pre-operative imaging procedures.

The medical image resulting from the patient's medical imaging scan will show the patient's internal anatomy along with the opaque indicia from the localization aid 10 of the present invention. Upon removal of the localization aid 10 from the patient, a duplicate of the opaque indicia will be visible on the patient's skin as a result of the transfer of ink from the marking mechanism applied to the bottom side of the sheet. This visible marking on the patient's body may or may not be opaque to follow up scans. The medical professional can now correlate the reference pattern on the medical image to the reference pattern on the patient's body. This gives the professional the ability to pinpoint where on the patient's body a targeted interest lies directly within the patient's body, below the skin. The marks on the skin are temporary, but will withstand sterilization of the skin during pre-operative procedures. The comparison between the indicia on the image, which shows the tissue of interest relative to the reference marks, and the correlatable reference mark indicia printed on the patient's body, allows the medical professional to accurately determine where the target tissue is located and thus where, for example, incisions should be made. Palpation is typically used in some circumstances to verify target location. When the operation is completed, the medical professional may further use the indicia printed on the patient's body as a guide to suturing the skin to its original position. Thus, the medical professional realigns the indicia during suturing to bring the skin back to its original position.

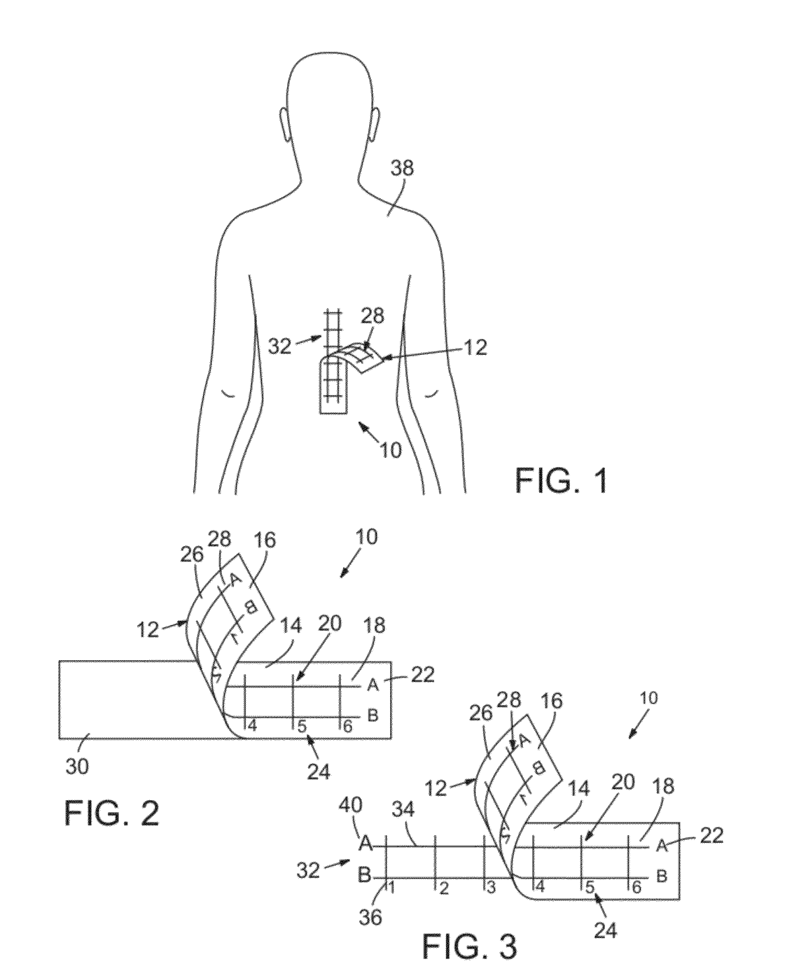

With reference now to the figures, as shown in FIGS. 2 and 3, the localizing aid 10 of the present invention comprises a flexible, non-opaque substrate sheet 12 having an upper or top surface 14 and opposed lower or bottom surface 16. The top surface 14 includes opaque indicia 18 that will appear visually on the medical imaging scan. The opaque indicia 18 may be in a locational reference pattern 20 such as the grid pattern shown in FIGS. 1, 2 and 3, which includes vertical and horizontal lines 22, or may take other forms such as dots, cross hatches, circles, graduated linear patterns, and combinations of any of these, or any other logical design that will assist with locating a precise area within a space. The locational reference pattern 20 may include reference labels, for example alpha, numeric or other symbols, identified generally in FIGS. 2 and 3 with reference number 24, or other symbols in any other area or direction. It will be appreciated that the combination of vertical and horizontal lines 22 and alpha and numeric symbols 24 are intended to assist the medical professional in accurately locating target tissue on the patient.

The bottom surface 16 of the sheet 12 includes an adhesive 26, which is preferably applied over the entire surface area of the bottom surface 16 to insure good adhesion between the sheet 12 and the patient when the sheet is applied to the patient's skin. The adhesive used is preferably a compound that results in minimal adverse reactions with most patients, and which also is easily released from the patient's skin when the aid 10 is removed.

Patient marking indicia shown generally at 28 is included on the bottom surface 16 of sheet 12 in the manner described below and in a duplicate or similar pattern to the locational reference pattern 20 on the top surface 14. A removable, protective backing sheet 30 is disposed over the adhesive 14 and patient marking indicia 28. The backing sheet 30 protects the adhesive 14 when the aid 10 is not being used.

As noted previously, the patient marking indicia 28 functions to transfer the pattern defined by the indicia onto the patient's skin. This is accomplished by using ink or other depositional material to define the reference pattern deposited on the bottom surface 16 of the sheet 12. There are numerous types of inks that are appropriate for use with the present invention and potential skin inks may include, but are not limited to, gentian violet, brilliant green and silver nitrate. Other types of materials may be included with the inks and incorporated therein to, for example, allow the ink that has been transferred to the patient to be opaque to subsequent medical imaging scans (and thus be visible on the images resulting from the scans). The ink is transferred directly from the sheet 12 to the patient's body by virtue of the ink or other marking material coming into direct contact with the patient's body when the localization aid is adhered to the body.

In FIG. 3 the localization aid 10 is schematically shown being removed from a patient's body to illustrate the patient marking indicia 28 transferred to the patient's skin. Thus, as the flexible sheet 12 is peeled upwardly and off the patient, the reference grid pattern defined by patient marking indicia 28 on sheet 12 has been transferred to the patient in an identical grid pattern, identified herein on the patient's body as body indicia 32. Body indicia 32 is a duplicate or is correlatable to patient marking indicia 28, and in the illustration of FIG. 3, includes the vertical and horizontal grid lines, identified with reference number 34 on the patient's body, and the alpha and numeric symbols identified on the patient's body with reference number 36.

FIG. 1 is similar to FIGS. 2 and 3 and shows localization aid 10 being removed from a patient 38. As noted, the sheet 12 is adhered to the patient 38 in the desired location and a medical imaging scan is performed—in the illustration of FIG. 1, the aid 10 is applied to the patient's back over the spine area. Thereafter, the sheet 12 is peeled away from the patient's back. As may be seen, the patient marking indicia 28, which in FIG. 1 comprises vertical and horizontal lines, is transferred onto the patient with identical or correlatable vertical and horizontal lines, resulting in an identical or correlatable grid pattern—body indicia 32—imprinted on the patient's skin.

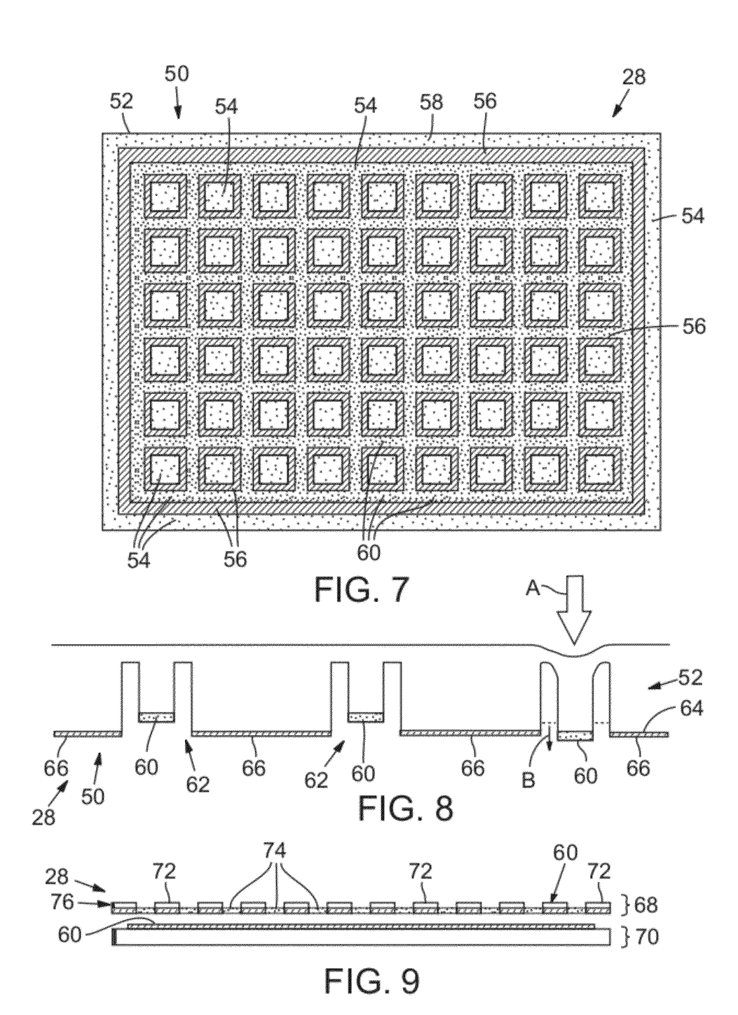

The patient marking indicia 28 may be configured in a variety of ways. Several illustrated and preferred embodiments are detailed below and in the accompanying drawings, but it is to be understood that the patient marking indicia used with the present invention is not limited to only these designs. The first embodiment of patient marking indicia 28 illustrated in FIG. 7 shows a cross-hatched grid pattern 50 formed on a thin, flexible substrate sheet 52. The grid pattern 50 is formed by embossing or debossing the substrate sheet 52 to create plural raised regions 54, and plural depressed regions 56 adjacent to the raised regions to define the individual portions of the grid. Adhesive 58, shown in dappled form in FIG. 7, is applied to the raised regions 54. Depositional material—that is, the ink used for patient marking indicia 28 (FIG. 2) is also layered on the flexible substrate, over the regions identified in FIG. 7 with reference number 60. Depressed regions 56 are located adjacent to the raised regions 54; however, the depressed regions create an analogous pattern to the locational reference pattern—in this image, a grid. Overall, this embodiment is like an ink stamp.

FIG. 8 is a cross-sectional view illustrating another embodiment of the patient marking indicia 28 and a pattern 50 created by embossing or debossing as detailed above, and which includes a further variation in the topography of a substrate sheet 52. In the embodiment of FIG. 8 the ink 60 or other depositional material that is to be transferred to the patient is deposited in a recessed area 62 that is set-back, inwardly from the bottom surface 64 of substrate sheet 52, as shown by the two recessed areas 62 shown on the left side of the illustration of FIG. 8. Once the substrate sheet 52 is applied to the patient, the ink 60 or other depositional material in recessed areas 62 would not immediately come into contact with the patient. However, when pressure is applied to the top surface of substrate sheet 52 as illustrated with arrow A in FIG. 8, the substrate sheet 52 flexes, causing the ink 60 in the recessed areas 62 to be moved toward the patient (arrow B, FIG. 8) into contact with the patient and thereby imprints the ink 60 onto the patient via direct contact. The embodiment just described and shown in FIG. 8 may be contrasted with the embodiment illustrated in FIG. 7 and described above, where the ink 60 or other depositional material is in immediate contact with the surface of the patient's body as soon as the sheet 52 is applied to the patient, and the grid pattern 50 is transferred to the patient as soon as the substrate sheet 52 is applied to the patient—without pressure applied to the sheet.

Yet another embodiment of the patient marking indicia 28 is shown in FIG. 9. The indicia 28 in FIG. 9 comprises two sheets, an outer sheet 68 and an inner sheet 70. The outer sheet 68 is a thin, flexible sheet. An adhesive 72 is layered over the outer sheet 68, which is the surface of the sheet that comes into contact with the patient. The outer sheet 68 is patterned via cutouts or voids 74, which define the locational reference pattern; the voids 74 define “windows” in the outer sheet 68 in the shape of the locational reference pattern.

The inner sheet 70 is also thin flexible sheet and has an ink or depositional material 60 deposited or layered on it on the side of the sheet 70 that faces outer sheet 68 in the assembled embodiment. The ink 60 or other depositional material covers at least the areas immediately under the “windows” defined by the voids 74 formed in the outer sheet 68. With this embodiment of FIG. 9, when the aid is applied to the patient, the exposed surface 76 of outer sheet 68 adheres to the patient. When pressure is applied to the aid, the inner sheet 70 flexes and the depositional material 60 residing between the inner sheet 70 and the outer sheet 68 is pushed through the voids 74 and thereby comes into contact with the patient and transfers the locational reference pattern to the patient's skin.

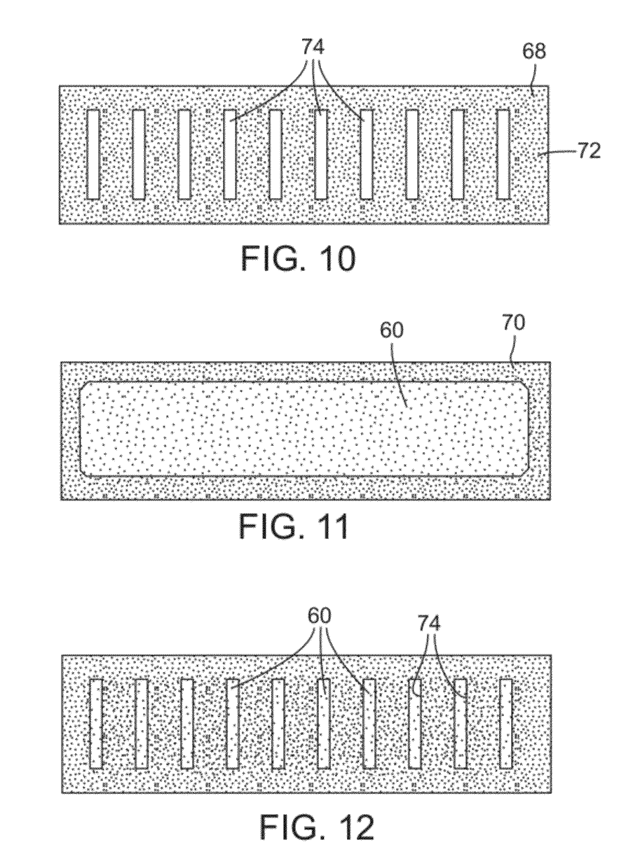

FIG. 10 illustrates a top view of the outer sheet 68 of the embodiment of FIG. 9, the outer sheet 68 shown in isolation without the inner sheet 70. The windows or voids 74 in the outer sheet 68 are analogous to the locational reference pattern—in this image, ruler markings.

FIG. 11 illustrates a top view of inner sheet 68 of the embodiment of FIG. 9, showing the inner sheet 70 in isolation without the outer sheet 68. Ink or other depositional material 60 is shown in dappled form and covering the area that would lie beneath the voids 74 in the outer sheet 68 when the outer and inner sheets are combined.

FIG. 12 illustrates the top view of the outer sheet 68 and inner sheet 70 assembled together as shown in FIG. 9. In FIG. 12 the ink 60 appears in dappled form through the windows defined by voids 74.

It will be apparent from the foregoing description and the drawing figures that the lower surface 16 of localization aid 10 may be configured in a variety of different ways. In a first embodiment, the lower surface is a planar surface onto which the patient marking indicia 28 is applied—when the aid 10 is applied to the patient's skin, the patient marking indicia is in immediate direct contact with the skin and is immediately transferred from the sheet 12 to the patient. In a second embodiment, the lower surface has an outer surface at a first plane onto which adhesive may be applied, and plural recessed regions that define the reference pattern and which are at a second plane relative to the first plane. The plural recessed portions carry the patient marking indicia, which is spaced apart from the patient's skin when the aid is applied to the patient. The patient marking indicia is in this instance transferred to the patient by applying pressure to the aid.

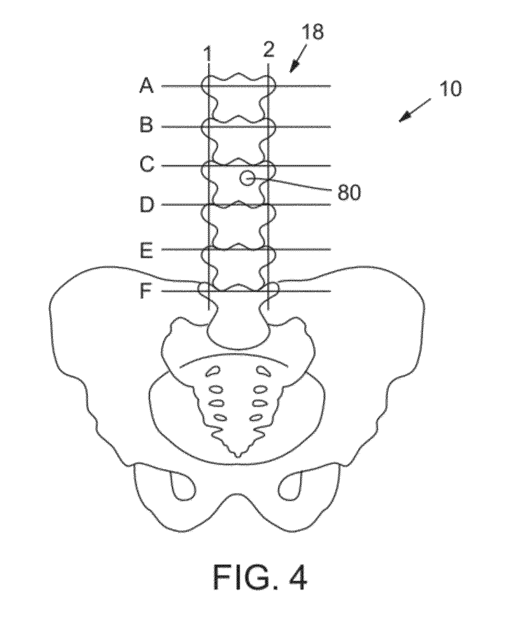

Reference is now made to the illustrations of FIGS. 4, 5 and 6 to describe use of the localization aid 10 according to the present invention. FIG. 4 is a schematic view of a medical image generated from a medical scanning procedure using the localization aid 10 as described herein. In FIG. 4, the lumbar portion of a patient's spine and the patient's hips are schematically shown as they might appear in an x-ray image. In preparation of the patient before a medical imaging scan, the localization aid 10 of an appropriate size and with appropriate localizational reference patterns are placed on the patient as described above. In the illustration of FIG. 4, the localization aid would be a relatively large sheet since it covers a significant area on the lumbar portion and hip portions of the patient's back. Thus, with the removable backing sheet 30 removed to expose the adhesive 26 and patient marking indicia 28, the sheet 12 is applied to the patient. With the sheet 12 adhered to the patient, the area is then scanned with the medical imaging scanner.

The resulting scan, shown in FIG. 4, clearly shows the opaque indicia 18, including in this instance grid lines and both alpha and numeric characters. The scan image also shows the internal anatomy of the patient's body as shown, and in FIG. 4 a target area is identified with reference number 80. The target area could be soft tissue, or in this case a portion of a vertebra located between the grid lines labeled C and D, and between the transverse grid lines 1 and 2.

Upon the removal of the localization aid 10 from the patient's body after the imaging scan has been completed, the patient marking indicia will cause an imprint of the identical or correlatable grid to be printed on the patient's body in the identical position as shown in the scan image, including all of the same grid lines and alpha and numeric characters in the same locations. The medical professional will refer to the imaging scan of FIG. 4 to locate the target area 80. The professional will then correlate the location of target area from the scan and recognize that the surgical or other procedure must be performed in the identical location on the patient—the patient marking indicia printed on the patient's back guides the professional to the precise location where the procedure must be performed.

As noted, the ink or other depositional material may incorporate compounds making the ink opaque to follow up scans. In other cases, and in some other procedures, follow up scans may require that the scan image be unobstructed with localizing grid lines and the like. In this case, depositional material will not be opaque to the scanning radiation.

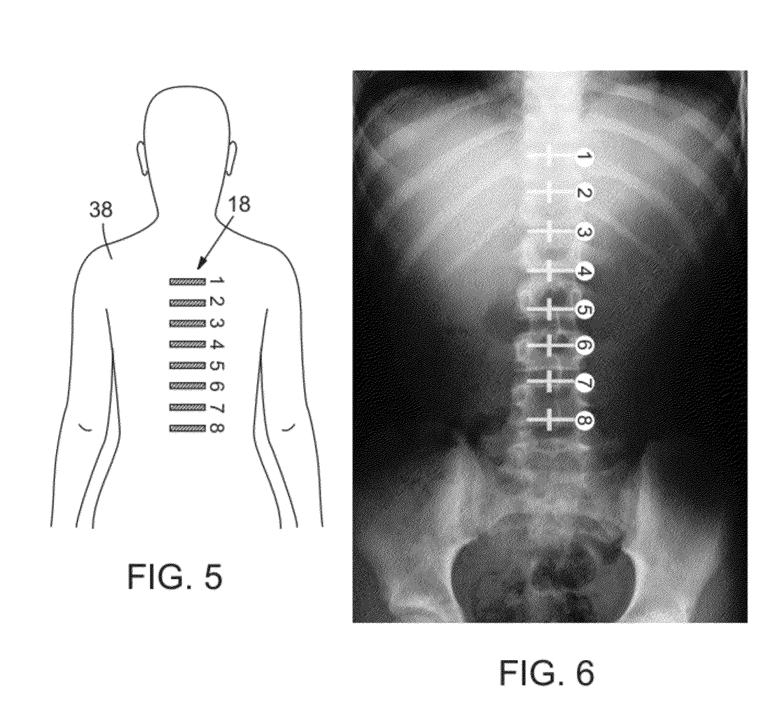

FIG. 5 shows a patient on whom the localization aid 10 has been used prior to an imaging scan, with the patient grid 32 derived from the patient marking indicia 28 clearly printed on the patient's back. FIG. 6 is the corresponding medical image generated with the localization aid in position on the patient's back. As may be seen, the image of FIG. 6 clearly shows the same pattern and characters as are printed on the patient's back in FIG. 5.

In addition to aiding a medical professional in locating a target in a patient, the patient grid 32 serves as a reference for accurate closure and suturing of an incision. Thus, once a procedure has been performed, the surgeon may use the patient grid to identify tissue locations on both sides of the incision that should be matched and sutured to facilitate accurate suturing of the wound.

While the present invention has been described in terms of a preferred embodiment, it will be appreciated by one of ordinary skill that the spirit and scope of the invention is not limited to those embodiments, but extend to the various modifications and equivalents as defined in the appended claims.

1. A medical procedure localization aid, comprising:
a substrate having an upper surface and an opposed lower surface, reference indicia on the upper surface comprising material opaque to medical imaging scans and arranged on the upper surface in a locational reference pattern, patient marking indicia on the lower surface, said patient marking indicia configured in a reference pattern corresponding to said locational reference pattern, and said patient marking indicia comprising material capable of transfer from said substrate to a patient.

2. The medical procedure localization aid according to claim 1 wherein the lower surface defines an outer surface at a first plane and the reference pattern on said lower surface comprises plural recessed regions at a second plane recessed into the flexible substrate relative to the first plane, and wherein the patient marking indicia is in the plural recessed regions.

3. The medical procedure localization aid according to claim 2 including an adhesive on the outer surface.

4. The medical procedure localization aid according to claim 3 wherein when the substrate is adhered to a patient the outer surface is in contact with the patient but the plural recessed regions are spaced apart from and not in direct contact with the patient.

5. The medical procedure localization aid according to claim 4 wherein pressure applied to the substrate causes the plural recessed regions to make direct contact with the patient to thereby transfer the patient marking indicia to the patient.

6. The medical procedure localization aid according to claim 1 wherein the reference indicia on the upper surface is radiopaque to x-radiation.

7. The medical procedure localization aid according to claim 1 wherein the reference indicia is a non-magnetic hydrogel.

8. The medical procedure localization aid according to claim 1 wherein the patient marking indicia is transparent to medical imaging radiation.

9. The medical procedure localization aid according to claim 1 wherein the patient marking material is opaque to medical imaging radiation.

10. The medical procedure localization aid according to claim 1 in which the locational reference pattern includes grid lines.

11. The medical procedure localization aid according to claim 10 in which the locational reference pattern includes alpha or numeric characters.

12-14. (canceled)

15. A medical procedure localization aid, comprising:
a flexible substrate having a first surface and an opposed second surface;
medical image reference indicia means on the first surface comprising material opaque to medical imaging, said medical image reference indicia arranged in a first reference pattern;
patient marking indicia on the second surface comprising material transferable from said flexible substrate to a patient's skin when said patient marking indicia is in direct contact with the patient's skin, said patient marking indicia arranged in a second reference pattern.

16. The medical procedure localization aid according to claim 15 in which the second reference pattern correlates to the first reference pattern.

17. The medical procedure localization aid according to claim 16 in which the second reference pattern is identical to the first reference pattern.

18. The medical procedure localization aid according to claim 15 wherein the second surface has an outer surface configured for making direct contact with the patient's skin and plural recessed portions defining the second reference pattern, said plural recessed portions spaced apart from the patient's skin when the localization aid is applied to the patient.

19. The medical procedure localization aid according to claim 18 wherein the patient marking indicia is confined to the plural recessed portions.

20. The medical procedure localization aid according to claim 19 wherein the localization aid is applied to the patient and pressure is applied to the localization aid the patient marking indicia is moved into direct contact with the patient to thereby transfer the patient marking indicia to the patient. Please add the following new claims:

21. A medical procedure localization aid, comprising:
a flexible substrate having opposed upper and lower surfaces;
reference indicia on the upper surface, said reference indicia opaque to medical imaging scans and arranged on the upper surface in a reference pattern;
patient marking indicia on the lower surface, said patient marking indicia transferable from said substrate onto a patient onto whom said flexible substrate has been applied.

22. The medical procedure localization aid according to claim 12 in which the patient marking indicia is configured in a reference pattern and the reference pattern of the patient marking indicia is aligned with the reference pattern of the reference indicia.

23. The medical procedure localization aid according to claim 13 wherein the lower surface defines an outer surface at a first plane and the reference pattern on said lower surface comprises plural recessed regions at a second plane recessed into the flexible substrate relative to the first plane, and wherein the patient marking indicia is in the plural recessed regions
